(19) United States
(12) Patent Application Publication
(10) Pub. No.: US 2019/0298298 A1
(43) Pub. Date: Oct. 3, 2019

(54) ULTRASOUND IMAGING METHOD

(71) Applicants: Meng-Lin Li, Hsinchu City (TW); QISDA CORPORATION, Taoyuan City (TW)

(72) Inventors: Meng-Lin Li, Hsinchu City (TW); Fu-Yen Kuo, Hsinchu City (TW); Tang-Chen Chang, Miaoli County (TW)

(21) Appl. No.: 16/271,870

(22) Filed: Feb. 10, 2019

(30) Foreign Application Priority Data
Mar. 29, 2018 (TW) 107110897

(51) Int. Cl. A61B 8/06; A61B 8/00; A61B 8/08; A61B 5/00

(52) U.S. Cl. CPC A61B 8/06; A61B 8/463; A61B 8/5246; A61B 8/0891; A61B 8/5269; A61B 8/488; A61B 5/7267

(57) ABSTRACT

An ultrasound imaging method includes steps of transmitting a plurality of ultrasound signals by a pulse repetition interval; receiving a plurality of reflected signals of the ultrasound signals; separating a blood flow signal and a clutter signal from the reflected signals by a neural network; calculating a blood flow parameter according to the blood flow signal; determining a blood vessel position according to the blood flow parameter; and adjusting an image signal corresponding to the reflected signals according to the blood flow parameter and the blood vessel position to generate an ultrasound image.

### BACKGROUND OF THE INVENTION

### 1. Field of the Invention

The invention relates to an ultrasound imaging method and, more particularly, to an ultrasound imaging method adapted to detect blood flow.

### 2. Description of the Prior Art

Since ultrasound scanning equipment does not destroy material structure and cell, the ultrasound scanning equipment is in widespread use for the field of material and clinical diagnosis. In general, color Doppler ultrasound and power Doppler ultrasound are usually used to detect blood flow in clinical diagnosis. However, the detection of blood flow is always affected by the disturbance of human tissue, such that the accuracy of the detection is reduced. At present, color Doppler ultrasound and power Doppler ultrasound of the prior art use a wall filter or an adaptive wall filter to separate a blood flow signal and a clutter signal generated by the disturbance of the tissue. However, for the variation of tiny blood flow, the band distribution of the blood flow signal overlaps the band distribution of the clutter signal, such that the wall filter cannot separate the blood flow signal and the clutter signal effectively. Consequently, the tiny blood flow cannot be detected. Furthermore, some prior arts use singular value decomposition (SVD) to analyze signals to separate the blood flow signal and the clutter signal effectively. However, SVD requires complicated matrix calculation, such that the hardware is hard to be implemented due to huge calculation.

### SUMMARY OF THE INVENTION

An objective of the invention is to provide an ultrasound imaging method adapted to detect blood flow, so as to solve the aforesaid problems.

According to an embodiment of the invention, an ultrasound imaging method comprises steps of transmitting a plurality of ultrasound signals by a pulse repetition interval; receiving a plurality of reflected signals of the ultrasound signals; separating a blood flow signal and a clutter signal from the reflected signals by a neural network; calculating a blood flow parameter according to the blood flow signal; determining a blood vessel position according to the blood flow parameter; and adjusting an image signal corresponding to the reflected signals according to the blood flow parameter and the blood vessel position to generate an ultrasound image.

According to another embodiment of the invention, an ultrasound imaging method comprises steps of transmitting a plurality of ultrasound signals by a pulse repetition interval; receiving a plurality of reflected signals of the ultrasound signals; separating a blood flow signal and a clutter signal from the reflected signals; calculating a blood flow speed according to the blood flow signal; determining a blood vessel position according to the blood flow speed; adjusting the pulse repetition interval according to the blood flow speed and/or adjusting a signal processing range corresponding to the reflected signals according to the blood vessel position; and adjusting an image signal corresponding to the reflected signals according to the blood flow speed and the blood vessel position to generate an ultrasound image.

As mentioned in the above, the invention replaces the wall filter or the adaptive wall filter of the prior art by the neural network to separate the blood flow signal and the clutter signal generated by the disturbance of the tissue. Accordingly, the invention can reduce the difficulty in implementing the hardware effectively. Furthermore, the invention may adjust the pulse repetition interval according to the blood flow speed and/or adjust a signal processing range corresponding to the reflected signals according to the blood vessel position. Therefore, the invention can optimize system parameter to improve efficiency and accuracy of detecting blood flow.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a flowchart illustrating an ultrasound imaging method according to an embodiment of the invention.

FIG. 2 is a schematic diagram illustrating a blood flow signal and a clutter signal separated from the reflected signals of the ultrasound signals by a neural network.

FIG. 3 is a flowchart illustrating an ultrasound imaging method according to another embodiment of the invention.

FIG. 4 is a flowchart illustrating an ultrasound imaging method according to another embodiment of the invention.

### DETAILED DESCRIPTION

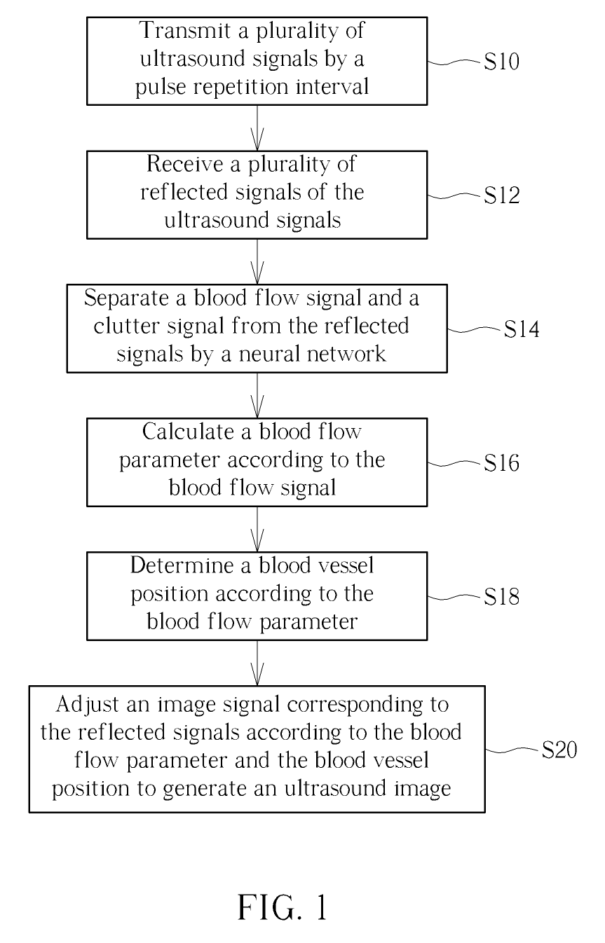

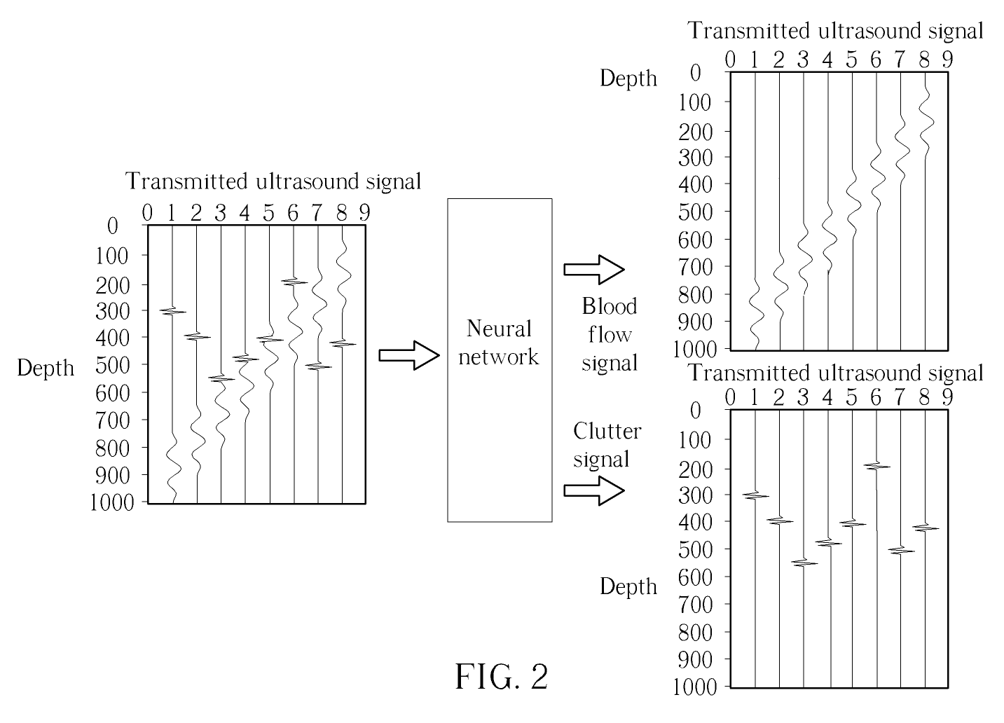

Referring to FIGS. 1 and 2, FIG. 1 is a flowchart illustrating an ultrasound imaging method according to an embodiment of the invention and FIG. 2 is a schematic diagram illustrating a blood flow signal and a clutter signal separated from the reflected signals of the ultrasound signals by a neural network. The ultrasound imaging method shown in FIG. 1 is adapted to color Doppler ultrasound and power Doppler ultrasound and used to detect blood flow to generate an ultrasound image.

When performing ultrasound scanning for a target object (not shown), an operator may operate an ultrasound probe (not shown) to transmit a plurality of ultrasound signals by a pulse repetition interval (PRI) (step S10 in FIG. 1) and receive a plurality of reflected signals of the ultrasound signals from the target object (step S12 in FIG. 1). Then, as shown in FIG. 2, the invention uses a neural network to separate a blood flow signal and a clutter signal from the reflected signals (step S14 in FIG. 1). In this embodiment, the aforesaid neural network may be a convolution neural network (CNN) or the like.

In this embodiment, the neural network has been trained for separating the blood flow signal and the clutter signal from the reflected signals of the ultrasound signals. The invention may prepare a plurality of training samples in advance, wherein each of the training samples comprises the reflected signals of the ultrasound signals shown in FIG. 2 and comprises the blood flow signal and the clutter signal separated from the reflected signals of the ultrasound signals. Then, the training samples are inputted into the neural network to train the neural network to separate the blood flow signal and the clutter signal from the reflected signals of the ultrasound signals. It should be noted that the detailed training process of the neural network is well known by one skilled in the art, so it will not be depicted herein in detail. Furthermore, for the neural network capable of supplying high complicated calculation, the invention may add characteristics between every two adjacent scanning lines and characteristics between different images for purposes of signal analysis and capture, so as to improve the recognition of the blood flow signal and the clutter signal.

After obtaining the blood flow signal, the invention may calculate a blood flow parameter according to the blood flow signal (step S16 in FIG. 1), wherein the blood flow parameter may be a blood flow speed or a signal intensity of the blood flow signal. If the ultrasound imaging method of the invention is applied to color Doppler ultrasound, the aforesaid blood flow parameter may be the blood flow speed. It should be noted that the method of calculating the blood flow speed according to the blood flow signal is well known by one skilled in the art and the details may be referred to “C. Kasai, K. Namekawa, A. Koyano, and R. Omoto, Real-Time Two Dimensional Blood Flow Imaging Using an Autocorrelation Technique, IEEE Trans. Sonics Ultrasonics, vol. SU-32, pp. 458-464, 1985.”, so it will not be depicted herein. Furthermore, if the ultrasound imaging method of the invention is applied to power Doppler ultrasound, the aforesaid blood flow parameter may be the signal intensity of the blood flow signal. It should be noted that the method of calculating the signal intensity of the blood flow signal according to the blood flow signal is also well known by one skilled in the art, so it will not be depicted herein.

After obtaining the blood flow parameter, the invention may determine a blood vessel position according to the blood flow parameter (step S18 in FIG. 1). It should be noted that the method of determining the blood vessel position according to the blood flow parameter is well known by one skilled in the art and the details may be referred to “Efficient Implementation of Ultrasound Color Doppler Algorithms on Texas Instruments' C64x™ Platforms”, so it will not be depicted herein.

Then, the invention may adjust an image signal corresponding to the reflected signals according to the blood flow parameter and the blood vessel position to generate an ultrasound image (step S20 in FIG. 1). In this embodiment, the invention may generate a black-and-white ultrasound image according to the reflected signals, wherein the black-and-white ultrasound image is generated by B mode. At the same time, the invention may adjust a color parameter corresponding to the blood flow signal according to the blood flow parameter and the blood vessel position and then generate a color ultrasound image, wherein the blood vessel position is labeled in the color ultrasound image by a color parameter corresponding to the blood flow parameter. Then, the invention may combine the color ultrasound image and the black-and-white ultrasound image to form the aforesaid ultrasound image.

Since the invention replaces the wall filter or the adaptive wall filter of the prior art by the neural network to separate the blood flow signal and the clutter signal generated by the disturbance of the tissue, the invention can reduce the difficulty in implementing the hardware effectively.

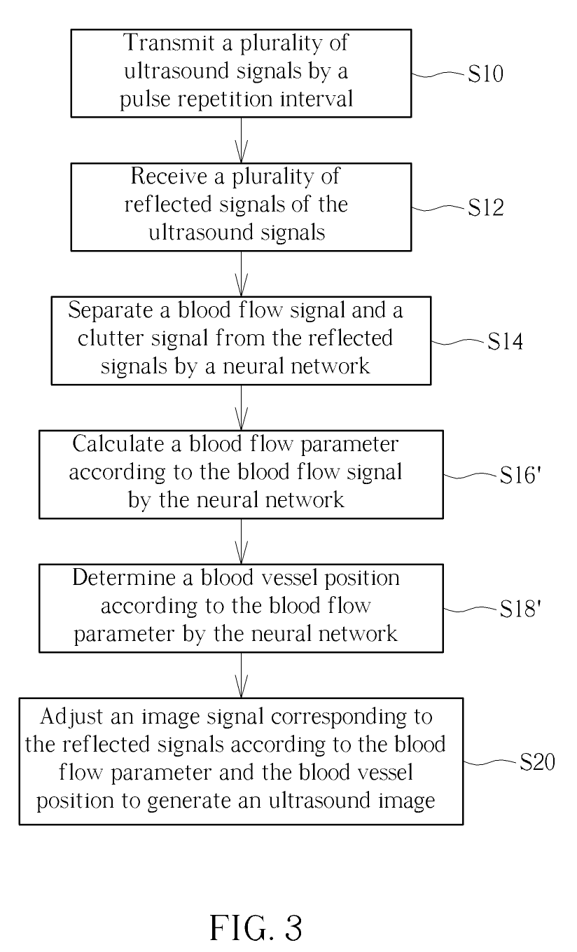

Referring to FIG. 3, FIG. 3 is a flowchart illustrating an ultrasound imaging method according to another embodiment of the invention. The main difference between the ultrasound imaging method shown in FIG. 3 and the ultrasound imaging method shown in FIG. 1 is that the step S16′ of the ultrasound imaging method shown in FIG. 3 uses the neural network to calculate the blood flow parameter according to the blood flow signal and the step S18′ of the ultrasound imaging method shown in FIG. 3 uses the neural network to determine the blood vessel position according to the blood flow parameter. In other words, the ultrasound imaging method shown in FIG. 3 uses the neural network to separate the blood flow signal and the clutter signal from the reflected signals, calculate the blood flow parameter according to the blood flow signal, and determine the blood vessel position according to the blood flow parameter. In this embodiment, the invention may prepare a plurality of training samples in advance, wherein each of the training samples comprises pattern samples of the blood flow signal and the clutter signal corresponding to 256 gray scale color mapping in Doppler shift frequency. Then, the training samples are inputted into the neural network to train the neural network. It should be noted that the detailed training process of the neural network is well known by one skilled in the art, so it will not be depicted herein in detail.

When the aforesaid neural network is a convolution neural network and the blood flow parameter is a blood flow speed, the ultrasound imaging method of the invention may further adjust at least one of the pulse repetition interval and the kernel size of the convolution neural network according to the blood flow speed, so as to improve efficiency and accuracy of detecting blood flow. For example, when the blood flow speed is fast, the pulse repetition interval may decrease correspondingly; when the blood flow speed is slow, the pulse repetition interval may increase correspondingly. For example, when the blood flow speed is fast, the kernel size may decrease correspondingly; when the blood flow speed is slow, the kernel size may increase correspondingly. It should be noted that the kernel size is preset by the convolution neural network for purposes of training and recognition. Since the principle of the kernel size of the convolution neural network is well known by one skilled in the art, it will not be depicted herein.

Moreover, the ultrasound imaging method of the invention may further adjust a signal processing range of a next ultrasound image according to the blood vessel position. For further illustration, when the blood vessel position of an i-th ultrasound image is known, the invention may adjust the signal processing range of an (i+1)-th ultrasound image (i.e. the next ultrasound image of the i-th ultrasound image) to cover the blood vessel position of the i-th ultrasound image, such that the invention need not to process the signals of non-blood vessel position of the i-th ultrasound image. Accordingly, the invention can reduce calculation effectively.

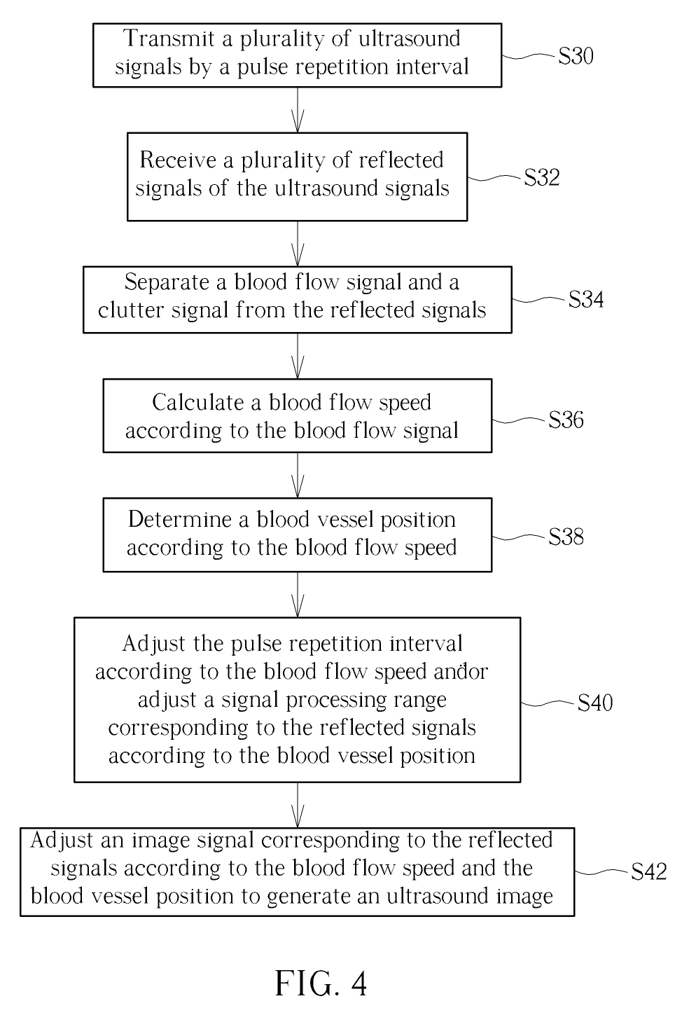

Referring to FIG. 4, FIG. 4 is a flowchart illustrating an ultrasound imaging method according to another embodiment of the invention. The ultrasound imaging method shown in FIG. 4 is adapted to color Doppler ultrasound and used to detect blood flow to generate an ultrasound image.

When performing ultrasound scanning for a target object (not shown), an operator may operate an ultrasound probe (not shown) to transmit a plurality of ultrasound signals by a pulse repetition interval (PRI) (step S30 in FIG. 4) and receive a plurality of reflected signals of the ultrasound signals from the target object (step S32 in FIG. 4). Then, the invention separates a blood flow signal and a clutter signal from the reflected signals (step S34 in FIG. 4). In this embodiment, the invention may use a neural network, a wall filter or an adaptive wall filter to separate the blood flow signal and the clutter signal from the reflected signals.

After obtaining the blood flow signal, the invention may calculate a blood flow speed according to the blood flow signal (step S36 in FIG. 4). It should be noted that the method of calculating the blood flow speed according to the blood flow signal is well known by one skilled in the art and the details may be referred to “C. Kasai, K. Namekawa, A. Koyano, and R. Omoto, Real-Time Two Dimensional Blood Flow Imaging Using an Autocorrelation Technique, IEEE Trans. Sonics Ultrasonics, vol. SU-32, pp. 458-464, 1985.”, so it will not be depicted herein.

After obtaining the blood flow speed, the invention may determine a blood vessel position according to the blood flow speed (step S38 in FIG. 4). It should be noted that the method of determining the blood vessel position according to the blood flow speed is well known by one skilled in the art and the details may be referred to “Efficient Implementation of Ultrasound Color Doppler Algorithms on Texas Instruments' C64x™ Platforms”, so it will not be depicted herein.

Then, the invention may adjust the pulse repetition interval according to the blood flow speed and/or adjust a signal processing range corresponding to the reflected signals according to the blood vessel position (step S40 in FIG. 4), so as to improve efficiency and accuracy of detecting blood flow. It should be noted that the manner of adjusting the pulse repetition interval and the signal processing range has been mentioned in the above, so it will not be depicted herein again.

Then, the invention may adjust an image signal corresponding to the reflected signals according to the blood flow speed and the blood vessel position to generate an ultrasound image (step S42 in FIG. 4). In this embodiment, the invention may generate a black-and-white ultrasound image according to the reflected signals, wherein the black-and-white ultrasound image is generated by B mode. At the same time, the invention may adjust a color parameter corresponding to the blood flow signal according to the blood flow speed and the blood vessel position and then generate a color ultrasound image, wherein the blood vessel position is labeled in the color ultrasound image by a color parameter corresponding to the blood flow speed. Then, the invention may combine the color ultrasound image and the black-and-white ultrasound image to form the aforesaid ultrasound image.

In another embodiment, the invention may use a convolution neural network to separate the blood flow signal and the clutter signal from the reflected signals, use the convolution neural network to calculate the blood flow speed according to the blood flow signal, and/or use the convolution neural network to determine the blood vessel position according to the blood flow speed. At this time, the convolution neural network may preset a kernel size. It should be noted that the kernel size is preset by the convolution neural network for purposes of training and recognition. Since the principle of the kernel size of the convolution neural network is well known by one skilled in the art, it will not be depicted herein. Accordingly, after obtaining the blood flow speed, the blood flow speed may be used to adjust at least one of the pulse repetition interval and the kernel size of the convolution neural network, so as to improve efficiency and accuracy of detecting blood flow.

As mentioned in the above, the invention replaces the wall filter or the adaptive wall filter of the prior art by the neural network to separate the blood flow signal and the clutter signal generated by the disturbance of the tissue. Accordingly, the invention can reduce the difficulty in implementing the hardware effectively. Furthermore, the invention may adjust at least one of the pulse repetition interval and the kernel size of the convolution neural network according to the blood flow speed and/or adjust a signal processing range corresponding to the reflected signals according to the blood vessel position. Therefore, the invention can optimize system parameter to improve efficiency and accuracy of detecting blood flow.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An ultrasound imaging method comprising steps of:
transmitting a plurality of ultrasound signals by a pulse repetition interval;
receiving a plurality of reflected signals of the ultrasound signals;
separating a blood flow signal and a clutter signal from the reflected signals by a neural network;
calculating a blood flow parameter according to the blood flow signal;
determining a blood vessel position according to the blood flow parameter; and
adjusting an image signal corresponding to the reflected signals according to the blood flow parameter and the blood vessel position to generate an ultrasound image.

2. The ultrasound imaging method of claim 1, wherein the step of adjusting an image signal corresponding to the reflected signals according to the blood flow parameter and the blood vessel position to generate an ultrasound image comprises steps of:
generating a black-and-white ultrasound image according to the reflected signals;
adjusting a color parameter corresponding to the blood flow signal according to the blood flow parameter and the blood vessel position;
generating a color ultrasound image; and
combining the color ultrasound image and the black-and-white ultrasound image to form the ultrasound image.

3. The ultrasound imaging method of claim 1, wherein the blood flow parameter is a blood flow speed or a signal intensity of the blood flow signal.

4. The ultrasound imaging method of claim 1, wherein the ultrasound imaging method uses the neural network to calculate the blood flow parameter according to the blood flow signal.

5. The ultrasound imaging method of claim 1, wherein the ultrasound imaging method uses the neural network to determine the blood vessel position according to the blood flow parameter.

6. The ultrasound imaging method of claim 1, wherein the neural network is a convolution neural network, the convolution neural network presets a kernel size, the blood flow parameter is a blood flow speed, the ultrasound imaging method further comprises step of:
adjusting at least one of the pulse repetition interval and the kernel size of the convolution neural network according to the blood flow speed.

7. The ultrasound imaging method of claim 1, further comprising step of:
adjusting a signal processing range of a next ultrasound image according to the blood vessel position.

8. An ultrasound imaging method comprising steps of:
transmitting a plurality of ultrasound signals by a pulse repetition interval;
receiving a plurality of reflected signals of the ultrasound signals;
separating a blood flow signal and a clutter signal from the reflected signals;
calculating a blood flow speed according to the blood flow signal;
determining a blood vessel position according to the blood flow speed;
adjusting the pulse repetition interval according to the blood flow speed and/or adjusting a signal processing range corresponding to the reflected signals according to the blood vessel position; and
adjusting an image signal corresponding to the reflected signals according to the blood flow speed and the blood vessel position to generate an ultrasound image.

9. The ultrasound imaging method of claim 8, wherein the step of adjusting an image signal corresponding to the reflected signals according to the blood flow speed and the blood vessel position to generate an ultrasound image comprises steps of:
generating a black-and-white ultrasound image according to the reflected signals;
adjusting a color parameter corresponding to the blood flow signal according to the blood flow speed and the blood vessel position;
generating a color ultrasound image; and
combining the color ultrasound image and the black-and-white ultrasound image to form the ultrasound image.

10. The ultrasound imaging method of claim 8, wherein the ultrasound imaging method uses a convolution neural network to separate the blood flow signal and the clutter signal from the reflected signals.

11. The ultrasound imaging method of claim 8, wherein the ultrasound imaging method uses the convolution neural network to calculate the blood flow speed according to the blood flow signal.

12. The ultrasound imaging method of claim 10, wherein the ultrasound imaging method uses the convolution neural network to determine the blood vessel position according to the blood flow speed.

13. The ultrasound imaging method of claim 10, wherein the convolution neural network presets a kernel size, and the blood flow speed is used to adjust at least one of the pulse repetition interval and the kernel size of the convolution neural network.
